(19) United States
(12) Patent Application Publication
(10) Pub. No.: US 2013/0295687 A1
(43) Pub. Date: Nov. 7, 2013

(54) Novel Method of Isolation of TLR4 from Cell Lysates of Mononuclear Cells

(71) Applicants: Panda Santosh Kumar, Bhubaneswar (IN); Ravindran Balachandran, Bhubaneswar (IN)

(72) Inventors: Panda Santosh Kumar, Bhubaneswar (IN); Ravindran Balachandran, Bhubaneswar (IN)

(73) Assignee: INSTITUTE OF LIFE SCIENCES, Bhubaneswar (IN)

(21) Appl. No.: 13/876,551

(22) PCT Filed: Nov. 11, 2011
(86) PCT No.: PCT/IN2011/000780

§ 371 Date: May 15, 2013

(30) Foreign Application Priority Data
Sep. 15, 2011 (IN) 1213/KOL/07

(51) Int. Cl. C07K 14/705

(52) U.S. Cl. CPC C07K 14/70596
USPC 436/501

(57) ABSTRACT

A novel method of isolation of TLR4 from cell lysates of mononuclear cells is provided. The method includes: collecting bovine adult filarial parasites (Setaria digitata); preparing aqueous extracts of setaria digitata (FAg) to produce affinity purified filarial glycoprotein (AgW); isolating human PBMCs from heparinised venous blood samples; subjecting the isolated human PBMCs to the step of incubation to produce PBMC lysates; coupling the said AgW with CNBR activated sepharose; and loading the AgW coupled with sepharose and PBMC lysates to an affinity purification column to isolate the receptor recognizing AgW and detecting the reactivity of anti-receptor antibodies and anti-human TLR4 antibodies to the affinity purified receptor using peroxidase conjugated anti-rabbit IgG (in solidphase assay).

### FIELD OF INVENTION

This invention relates to a novel method of isolation of TLR4 from cell lysates of mononuclear cells.

### BACKGROUND OF THE INVENTION

TLR4 is the first characterized mammalian Toll expessed by a variety of cell types like macrophages and dendritic cells (Medzhitov, 2001) and responsible for LPS signaling during Gm-ve bacterial infection (Kawai et al, 2001, Kawai et al, 2011). LPS present in the cell wall of Gm-ve bacteria binds to LPS binding protein (O Neill et al, 2007). LPS-LBP complex binds to CD14, a glycosylphosphatidylinositol (GPI) linked protein expressed on the surface of phagocytes and form a ternary complex (Akira et al, 2006). The ternary complex then transfers LPS to MD2, a protein associated with TLR4 and LPS-MD2 complex induce signal via TLR4. LPS-TLR4 signalling resulted in production of inflammatory mediators like TNF-α, IL-1β, IL-6 and NO ect which play critical role in protective immunity against bacterial infection. Nitric oxide produced during LPS signaling is required for control of intracellular infections like Mycobacterium and Toxoplasma (E I Kasmi et al, 2008). Protection against Gm-ve bacteria was conferred by TLR4 signalling (Takeuchi et al, 1999). C3H/HeJ mice (TLR4 mutant) are more susceptible to Gm-ve bacterial infection (Netea et al, 2004, Branger et al, 2004). Later it was also demonstrated that absence of TLR4 signaling make prone to Salmonella peritonitis and Klebsiella pneumonia sepsis (Yang et al, 2002, Bernheiden et al, 2001, Fierer et al, 2002, Knapp et al, 2003). Innate immune activation by TLR antagonists is helpful in DC maturation and B cell activation; produce critical mediators working as anti-allergen, anti-cancer and anti-infection, whereas dysregulated innate immunity leads to development of autoimmunity, sepsis and atherosclerosis (Ishi et al, 2004, Eichacker et al, 2002, Phillip et al, 2004).

Immunomodulatory components from pathogens (PAMP) are known to induce effective host immune response through Pattern recognition receptors particularly TLRs but direct binding of PAMPs to TLRs remain contradictory-LPS signals through TLR4 but does not directly not bind to TLR4 (da silva et al, 2001). A secreted protein from human hook worm Necator americans (Hsieh et al, 2004), recombinant Ov-Asp-1 from Onchocerca volvulus (He et al, 2009) and trehalose dimycolate from M. tuberculosis (Ishikawa et al, 2009) are known to bind directly to antigen presenting cells but the receptors mediating this interaction are not known. Synthetic bacterial lipopeptide and peptidoglycan binding to extracellular domain of TLR2 has been demonstrated. (Vasselson et al, 2004, Iwaki et al, 2002) Another study by Melendez et al reported the direct binding of ES-62, a filarial glycoprotein to TLR4. We explore this possibility in our study and demonstrate AgW (affinity purified filarial glycoprotein) binds to TLR4 on the surface of human monocytes. AgW was coupled to CNBR activated sepharose and an affinity purification column was prepared through which TLR4 was affinity purified from human mononuclear cell lysates.

Conventionally recombinant TLR4 proteins are prepared by cloning TLR4 gene. TLR4 gene along with a tag sequence are cloned and expressed in bacteria but there is a limitation of proper folding of the protein in bacteria but here in this study we report a novel method by which functionally folded TLR4 can be isolated directly from the host membrane lysates by an affinity purification column.

### OBJECTS OF THE INVENTION

An object of the present invention is to propose a novel method of isolation of TLR4 from cell lysates of mononuclear cells;

Another object of the present invention is to propose a novel method of isolation of TLR4 by affinity purification column chromatography;

Further, object of the present invention is to propose a novel method by which functionally folded TLR4 can be isolated directly from the host membrane lysates by an affinity purification column.

### BRIEF DESCRIPTION OF THE INVENTION

According to this invention there is provided a novel method of isolation of TLR4 from cell lysates of mononuclear cells comprising:
Collecting bovine adult filarial parasites (Setaria digitata);
preparing aqueous extracts of setaria digitata (FAg) to produce affinity purified filarial glycoprotein (AgW);
isolating human PBMCs from heparinised venous blood samples;
subjecting the isolated human PBMCs to the step of incubation to produce PBMC lysates;
coupling the said AgW with CNBR activated sepharose;
loading the AgW coupled with sepharose in an affinity purification column incubating PBMC lysates in the loaded affinity purification column and isolating the receptor that binds to AgW.
developing anti-receptor antibodies in rabbits
detecting reactivity of anti-receptor antibodies and anti-human TLR4 antibodies to the affinity purified receptor using peroxidase conjugated anti-rabbit IgG (in solidphase assay).

### BRIEF DESCRIPTION OF THE ACCOMPANYING DRAWINGS

FIG. 1a: Human PBMCs were incubated with biotinylated AgW for 30 minutes at 4° C. followed by staining with Streptavidin-FITC and analysed by FACS. Binding of biotinylated reagents to monocyte populations are shown.

FIG. 1b: Human PBMCs were incubated with biotinylated anti human IgG or anti human IgM with and without AgW for 30 minutes at 4° C. followed by staining with Streptavidin-FITC and analysed by FACS. Binding of biotinylated reagents to monocyte populations are shown.

FIG. 1c: ELISA plates were coated with human IgG (purified from protein A sepharose column). After blocking with skimmed milk PBS, anti-receptor antibodies were incubated. Binding of anti receptor antibody was detected by using peroxidase conjugated anti-rabbit IgG. Anti-human IgG HRP was taken as a positive control. The enzyme activity was measured using OPD.

FIG. 2a: ELISA plates were coated with valine, serpin, alt-1, alt-2, ES antigens, L3 antigens, FAg and AgW incubated with biotinylated affinity purified receptors and probed with avidin peroxidase. The enzyme activity was measured using OPD.

FIG. 2b: ELISA plates were coated with different concentrations of AgW incubated with biotinylated affinity purified receptors and probed with avidin peroxidase. The enzyme activity was measured using OPD.

FIG. 3 shows that different concentrations of affinity purified receptors were coated in an ELISA plate and incubated with anti-rabbit TLR4 antibodies and probed with anti-rabbit IgG peroxidase. The enzyme activity was measured using OPD.

FIG. 4a: shows that Human PBMCs were incubated with rabbit anti-receptor antibodies with and without AgW and probed with anti-rabbit IgG FITC and analysed by FACS. Binding of both the antibodies to monocytes population is shown.

FIG. 4b: shows that Human PBMCs were incubated with rabbit anti-TLR4-PE antibodies with and without AgW and analysed by FACS. Binding of both the antibodies to monocytes population is shown.

FIG. 4c: Human PBMCs were incubated with rabbit anti-receptor antibodies and anti-TLR4 PE antibodies. The cells were then probed with anti-rabbit IgG FITC and analysed by FACS. Representative dots in the right upper quadrant showing the cells binding to both the antibodies.

FIG. 4d: Human PBMCs were incubated with anti-TLR4 PE antibodies with and without anti-receptor antibodies analysed by FACS.

FIG. 4e: Murine bone marrow macrophages were incubated with anti-receptor antibodies and probed with anti-rabbit IgG FITC and analysed by FACS.

FIG. 5a: Soluble TLR4 is recognized by anti-receptor antibodies in solidphase. Extracts of S. digitata, AgW, N. bracilliences, H. polygyrus were coated in an ELISA plate. After blocking with 1% skimmed milk-PBS, lysates of human PBMCs were incubated for 2 hr at 37° C. The plates were thoroughly washed and were incubated with anti-mouse TLR4 and anti-receptor antibodies and bound antibody was detected using peroxidase conjugated anti-rabbit IgG. The enzyme activity was measured using OPD. % mean±SEM of two individual experiments are shown.

FIG. 5b: Affinity purified receptor was separated in a non reducing gel and then transferred to a nitrocellulose membrane. The membrane was blocked using 5% Skimmed milk PBS and probed with anti-receptor antibodies followed by anti-rabbit IgG peroxidase. Enzyme activity was detected using DAB.

FIG. 6a,b: mouse (BALB/C) BMDMs were cultured in sterile Dulbecco's modified eagles medium containing 10% heat inactivated fetal calf serum in humidified atmospheric condition (5% CO₂,80% humidity). After 8-10 hrs incubation at 37° C. non adherent cells were removed by washing with sterile medium. The remaining adherent cells were stimulated with LPS with and without anti-receptor antibodies for 48 hrs. Culture supernatants were harvested and TNF-α, IL-1β was quantified according to the manufacturer's instruction and nitrite by Griess reagent.

FIG. 6c: BALB/C mice were injected IP with lethal dose of LPS (15 mg/Kg body wt) with or without anti-receptor antibodies (100 μm) and the mortality was scored. (n=10).

### DETAILED DESCRIPTION OF THE INVENTION

Collection of Bovine Adult Filarial Parasites (Setaria digitata)

Peritoneal dwelling adult female filarial parasites (Setaria digitata) were collected from cattle in a local abattoir, attached to the local zoological park at Nandankanan, Bhubaneswar after obtaining necessary approval from zoo authorities. The worms were transported to the laboratory in Dolbecco's Modified Eagles Medium (DMEM) (Sigma) pH 7.00 containing antibiotics [Penicillin Streptomycin solution 1 ml/100 ml of medium] (Sigma) and 1% glucose (Hi-media).

### Preparation of AgW and Labeling:

Aqueous extracts of S. digitata (designated as FAg) was prepared by homogenization followed by ultrasonication. One milligram of FAg was passed through WGA-Sepharose (Sigma) column and the unbound proteins were washed by passing PBS and glycoproteins bound to WGA were eluted by Glycine-HCl buffer (pH 3.6).The pH of the elutes was adjusted using 0.1 M NaOH and dialysed against PBS. Protein concentration of the elute (AgW) was estimated and stored at −20° C. for further use. AgW was biotinylated using appropriate derivatives of biotin i.e. N-hydroxysuccinamide derivatives (Sigma) suitable for protein labeling.

Lyophilized Brugia pahangi somatic extracts were kindly gifted by Dr. E. Devaney, University of Glasgow, UK and aqueous extracts of Nippostrongylus brasiliensis and Heligomosomoides polygyrus were gifted by Dr. Rick maizel, University of Edinburgh, UK.

### Animals

BALB/C mice were obtained from National Institute of Immunology, New Delhi which was originally imported from Jackson laboratories, Germany. Breeding and maintenance were done at the animal facility at Institute of Life Sciences, Bhubaneswar, India. 8-10 weeks old animals were used for this study. Institutional animal ethics committee of Institute of Life Sciences, Bhubaneswar approval was obtained for all the investigations conducted in mice.

### Bone Marrow Cells Culture.

Mouse bone marrow cells were collected from femoral shafts by flushing with 3 ml. of cold sterile DMEM (Sigma) supplemented with 20 mM of L-Glutamine (ICN), antibiotics (1 ml penicillin and streptomycin/100 ml of medium) (Sigma)containing 10% FBS. The cell suspension was passed through a sieve to remove large clumps. The cell suspension was washed 2-3 times with sterile DMEM and adjusted to 0.5×10⁶ cells/well and cultured in 24 well plates. After 8-10 hrs incubation at 37° C. non adherent cells were removed by washing with sterile DMEM and the adherent cells were stimulated with LPS (Sigma) with or without anti receptor antibodies at 10 μg/ml and 100 μg concentration respectively. After 48 hrs the supernatants were aspirated, frozen at −80° C. and used later for estimation of cytokine levels.

### Human Blood Samples

The study population was drawn from areas in and around Bhubaneswar, Orissa. About 5 ml. of nocturnal blood each was collected in heparinised glass vials from the selected human study population as per the ethical guidelines of ILS, Bhubaneswar, Orissa.

### Isolation of Human PBMCs and Culture

Human PBMCs were isolated from heparinised venous blood samples by density gradient centrifugation method using Histopaque (Sigma). Briefly, the heparinised blood was layered on LSM medium gently in the ratio of 1:1 and subjected to centrifugation at 1000 RPM for 30 minutes. The white layer representing PBMCs was aspirated out gently and transferred aseptically into sterile centrifuge tubes. The suspension of cells was then washed and used in binding study.

### Preparation of PBMC Lysates

Human PBMCs (2×10⁶) were incubated with 2 ml of cell lysis solution (Sigma) and a cocktail of protease inhibitors (Sigma) for one hr. at 4° C. and then ultra-sonicated. The supernatant was collected by centrifuging at 4000 RPM for 10 minutes and stored for further use.

Isolation of Receptor Recognizing AgW

AgW was coupled to CNBR activated sepharose and loaded in an affinity purification column and washed thoroughly to remove the uncoupled AgW. Human PBMC lysates were incubated in the column at 37° C. for 30 minutes and then the column was washed by PBS to remove the unbound proteins. The proteins bound to AgW column were eluted using Glycine HCl buffer (p^{H} 3.6). The p^{H} of the elutes was adjusted using 0.1 M NaOH and dialysed against PBS. The eluted protein concentration was measured and stored at −20° C. for further use.

### Preparation of Polyclonal Anti-Receptor Antibodies in Rabbits

A rabbit was immunized with 0.3 mg of affinity purified receptor with Complete Freunds Adjuvant subcutaneously. Three booster dosages were given. One is with incomplete freunds adjuvant intramuscularly and the second and third is without adjuvant, intra peritoneally. Pre bleed was collected from the animal prior to immunization. Serum was collected on day 55 and the titer of polyclonal antibody was checked in solid phase assay.

### Quantification of Cytokines

Supernatants from mouse BMDM cultures were analysed for levels of TNF-α, IL-1β by a sandwich ELISA according to manufacturer's instruction using commercially available ELISA kits (e-Biosciences).

### Nitrite Estimation

Nitrite concentration was estimated using Griess reagent. Culture supernatant or plasma was mixed with equal volume of Griess reagent (1% of sulfalinamide and 0.1% of N-(1-napthyi ethylenediamine in 2.5% H₃Po₄). After 15 minutes incubation at room temperature the nitrite concentration was measured at 540 nm using a microplate reader. Sodium nitrite (12.5-200 μm) was used as nitrite standards.

### Flow Cytometry

Human PBMCs or mouse BMDM (1×10⁶/ml) were stained for 30 minutes at 4° C. with fluorescence labeled antibodies specific to CD14, TLR4 (e Biosciences) or affinity purified receptors mixed with and without AgW along with relevant isotype controls. The cells were thoroughly washed to remove the unbound antibodies and analysed by FACS (BD FACS caliber).

### Enzyme Linked Immunosorbent Assay.

ELISA plates (Nunc maxisorp) were coated with 1 μg of PBS extracts of S. digitata, AgW, N. brasiliensis, H. polygyrus. After blocking with 1% skimmed milk-PBS (Hi-media), human PBMC lysates were incubated for 2 hr at 37° C. The plate was thoroughly washed and were incubated with rabbit anti-receptor antibodies and rabbit anti-human TLR4 (e Biosciences) antibodies. The binding of anti-receptor antibodies and anti-human TLR4 was detected by using peroxidase conjugated anti-rabbit IgG (Sigma). The enzyme activity was measured using OPD (Sigma).

### SDS Page and Western Bloting

Lysates of human PBMCs were separated on 10% polyacrylamide gels and transferred to nitrocellulose membranes (Millipore).The nitrocellulose membrane was blocked for overnight with 5% skimmed milk-PBS. The membrane was washed thoroughly for 5-6 times with PBS-tween and incubated with anti-receptor antibodies followed by anti-rabbit IgG peroxidase antibodies. The enzyme activity was detected using DAB.

### Mouse Model of Endotoxemia

8-10 weeks old BALB/C mice were intraperitoneally injected with 15 mg/Kg body wt LPS with control and anti receptor antibodies and observed for mortality a period over 168 hrs.

### AgW Binds to TLR4 on the Surface of Human Monocytes.

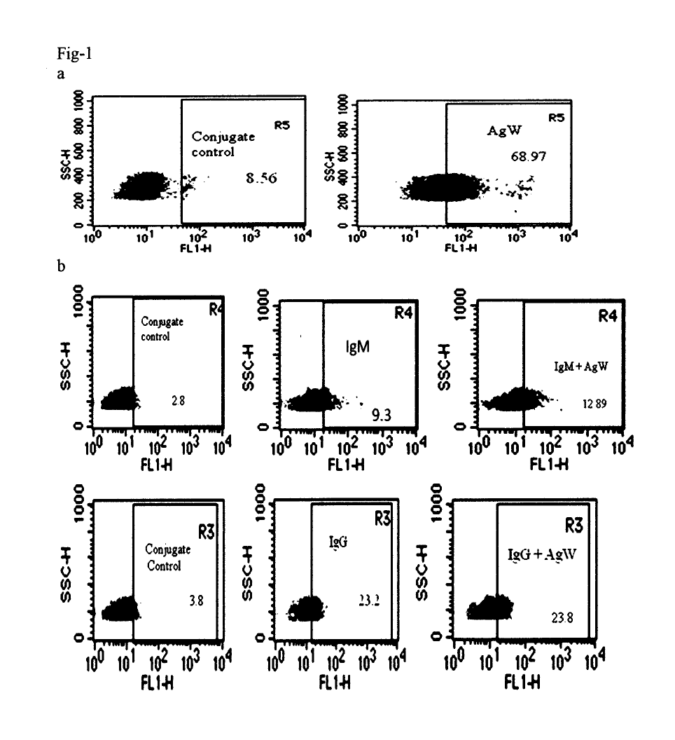

We tested the binding property of an affinity purified filarial glycoprotein (AgW) while searching for an elusive innate receptor for helminthes or helminth products. Biotinylated AgW directly bound to the surface of human monocytes (FIG. 1a). We exclude the possibility of binding of AgW to cytophillic antibodies on the cell surface by a competitive inhibition assay. AgW failed to inhibit the reactivity of anti-human IgG or anti-human IgM antibodies to the monocytes suggesting that AgW is not recognized by cytophillic antibodies on the surface of monocytes rather it is recognized by a receptor on the monocytes surface (FIG. 1b). A filarial antigen ES-62 is known to bind dendritic cells through TLR4, so we tested this possibility. Reactivity of anti TLR4 antibodies was inhibited by this binding suggesting that AgW bound to TLR4 on the surface of monocytes (FIG. 1c).

### Affinity Purified Receptor Binds to AgW on Solid Phase.

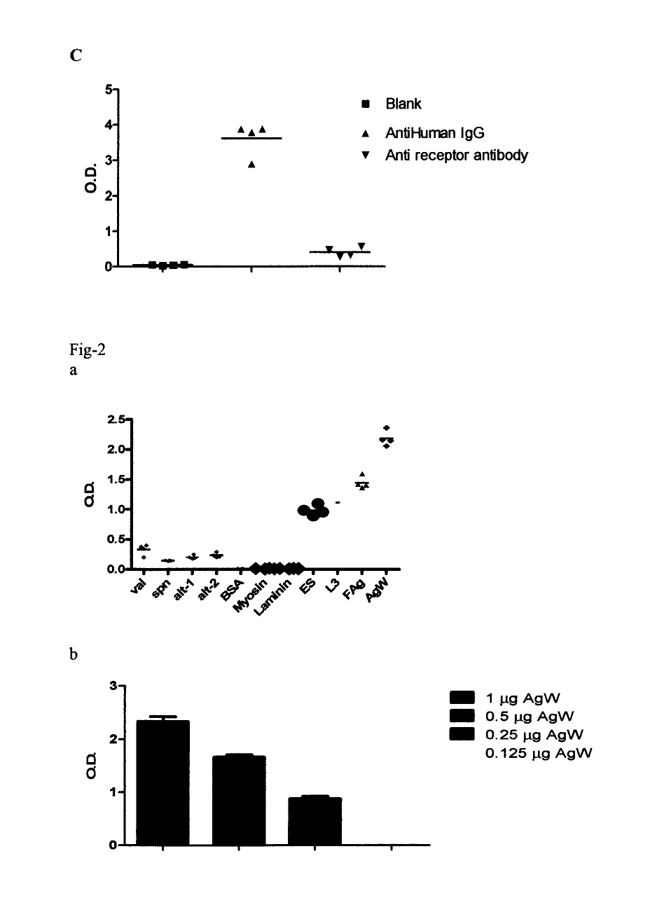

Using the binding property of AgW to monocytes we affinity purified the receptor and validated in solid phase assay. Biotinylated affinity purified receptor was demonstrated to bind to AgW on solidphase assay. ELISA plates were coated with both filarial and non filarial proteins and incubated with biotinylated receptor followed by streptavidin-peroxidase. It was observed that the affinity purified receptor binds to AgW containing filarial proteins whereas it fails to bind to control proteins like BSA, Myosin or laminin (FIG. 2a). Affinity purified receptor also bind AgW in a dose dependent manner revealing its specificity (FIG. 2b).

Affinity Purified receptor is TLR4.

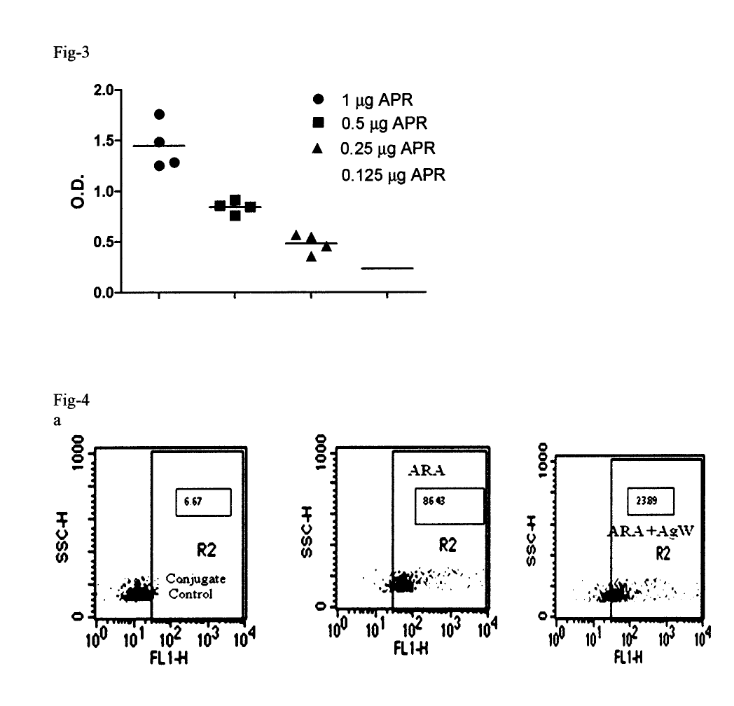

As it was demonstrated that AgW binds to TLR4 we tested this possibility. An ELISA plate was coated with affinity purified receptor followed by incubation with anti-TLR antibodies. Anti-TLR4 antibodies reacted with affinity purified receptor in a dose dependent manner suggesting that the affinity purified receptor is TLR4 (FIG. 3).

Reactivity of Anti-Receptor Antibody as well as Anti-TLR4 Antibodies to the Cell Surface is Inhibited by AgW.

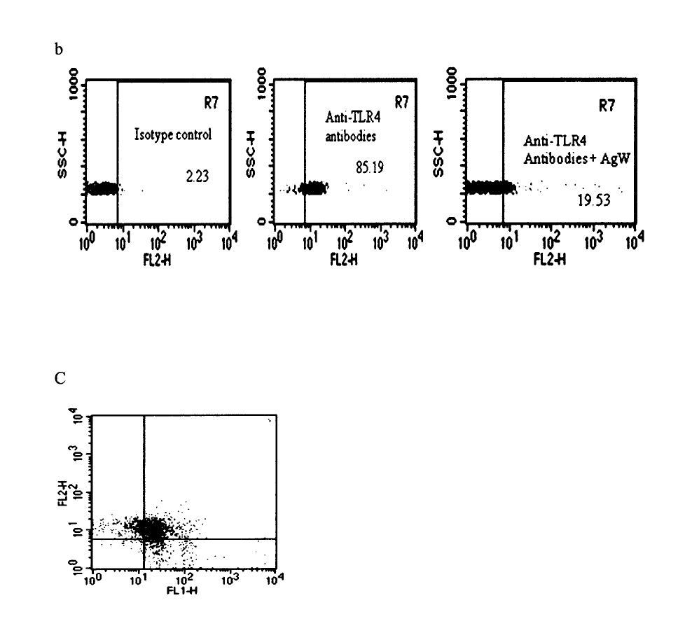

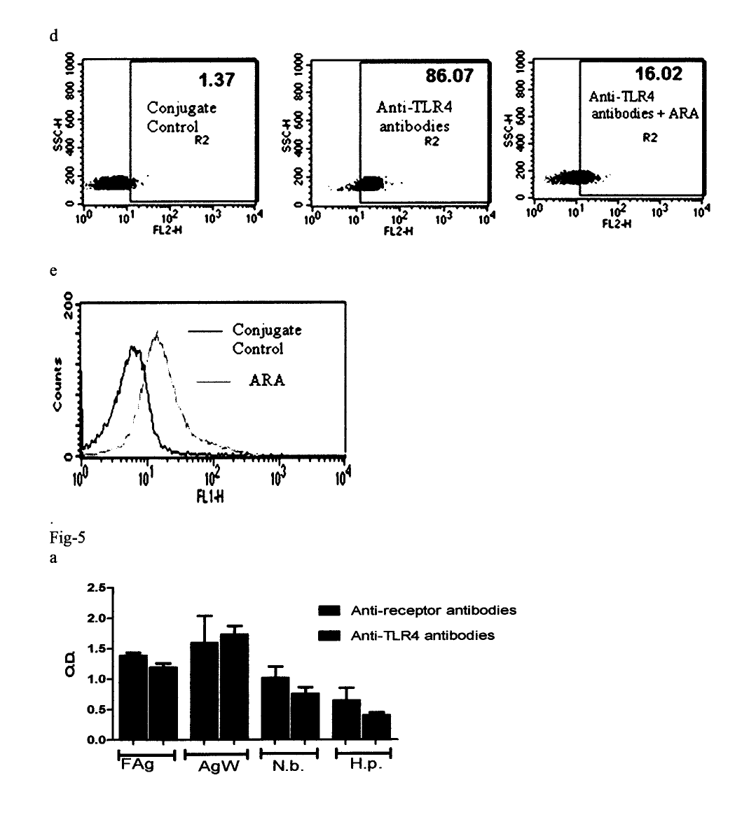

As part of the characterization process of the affinity purified receptor we raised a polyclonal antibody against the affinity purified receptor (anti-receptor antibodies) in rabbit and compare it with commercially available anti-TLR4 antibodies. Reactivity of both the antibodies to the surface of human monocytes was comparable. Further the reactivity of both the antibodies to human monocytes was inhibited by AgW (FIG. 4a,b). Both the antibodies bind to surface of the same population of monocytes (FIG. 4c) and reactivity of anti-TLR4 antibodies was inhibited by anti-receptor antibodies (FIG. 4d). Anti-receptor antibodies were also bind to murine bone marrow macrophages (FIG. 4e).

### Soluble TLR4 is Recognized by Anti-Receptor Antibodies.

The affinity of the anti-receptor antibody to TLR4 was validated in a solidphase assay. Different helminthic antigens were coated in an ELISA plate followed by incubation with membrane lysates of human PBMCs (containing TLR4) and then with both anti-TLR4 and anti-receptor antibodies. It was demonstrated that reactivity of both the antibodies are comparable (FIG. 5a). Further binding of anti-receptor antibodies to TLR4 was confirmed by western blotting. Soluble membrane lysates of human PBMCs were separated in a native SDS PAGE and then transferred to a nitrocellulose membrane. The nitrocellulose membrane was probed with anti-receptor antibodies and it was observed that anti-receptor antibodies bind to a protein of 90 KD mol wt. equivalents to the mol. wt. of TLR4 (FIG. 5b).

### LPS Induced Signaling and Endotoxic Shock was Inhibited by Anti-Receptor Antibodies.

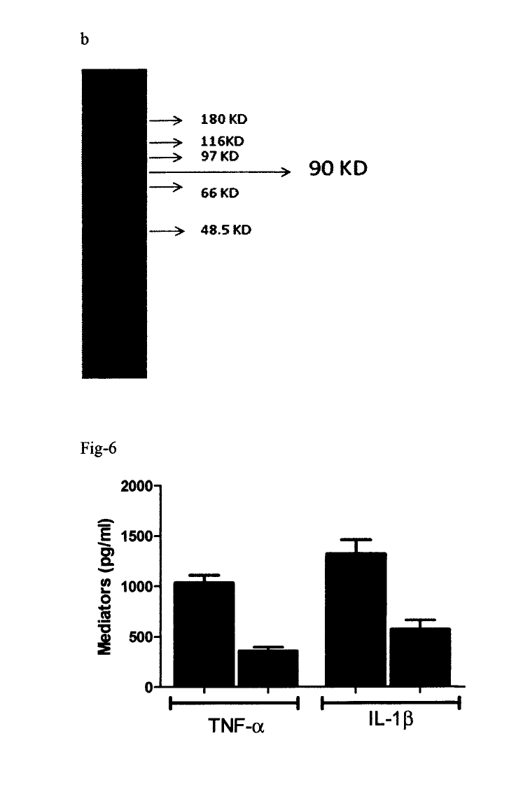

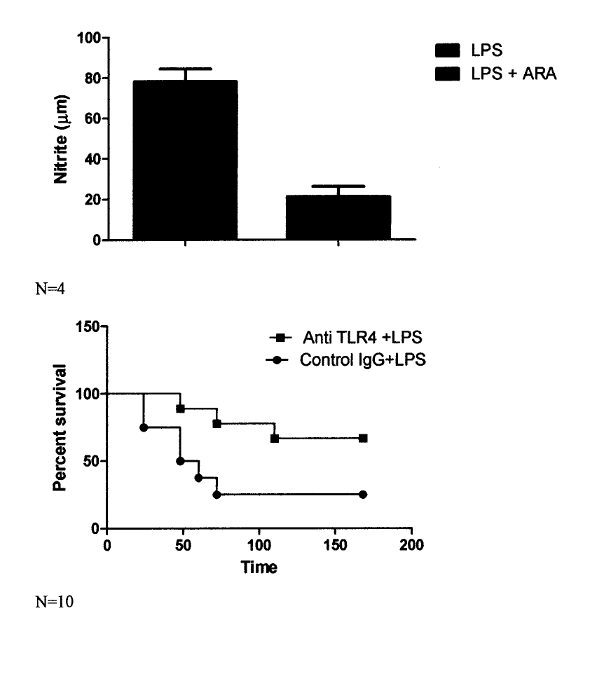

The role of anti-TLR4 or anti-TLR4/MD2 antibodies in blocking LPS induced signaling is well documented (Bruno et al, 2008, Thierry et al, 2009). We validated the efficiency of anti-receptor antibodies in blocking of LPS signaling. Murine bone marrow derived macrophages were stimulated with LPS with and without anti-receptor antibodies for 48 hrs. Levels of TNF-α, IL-1β and nitrite were quantified in the culture supernatants. It was observed that LPS induced mediators were inhibited by anti-receptor antibodies (FIG. 6a,b). We sought a direct in vivo validation of anti-receptor antibodies by administering it to a mouse model of endotoxemia. Co-administration of lethal dose of LPS and anti-receptor antibodies protected mice from endotoxic shock (FIG. 6b).

A simple and efficient method to isolate TLR4 from the cell lysates of human peripheral mononuclear cells. This procedure doesn't require complex equipments and skills and easier than tedious conventional methods that require recombinant DNA technology. Conventionally Fusion protein is prepared by cloning the TLR4 gene along with a tag sequence and allowed to express in bacteria. As the splicing and protein folding mechanism in bacteria is not so well equipped as eukaryotes, the functional folding of the protein along with the tag is doubtful. In this study we discovered a very rapid, convenient and easy method by means of which functionally folded TLR4 can be purified through an affinity purification column. The affinity purification column was prepared by coupling AgW with cyanogen bromide activated sepharose which is easier and required less skill than cloning and expressing a fusion protein. Further it is very less expensive than the costly molecular biological tools like plasmids or restriction enzymes. The column can be reused at least three times if proper storage care is taken.

This method of isolation will provide an ideal tool and will open new possibilities for the researchers who are unable to pursue their research on TLR4 due to lack of commercial reagents. As an instance our group (unpublished observations) established that gerbils are far more susceptible to endotoxic shock than Mastomys. 1/40 th of the lethal dose of Mastomys is enough to cause endotoxic shock and death in gerbils. The first question need to be answered in the above paradoxical models is whether differential expression of TLR4 takes place in both the species but it cannot be pursued due to lack of anti-gerbil/anti-Mastomys TLR4 antibodies. Gram negative bacterial infection, endotoxemia and sepsis is a common phenomenon in horse (Morris, 1991). Again the expression and function of TLR4 cannot be examined due to unavailability of commercial reagents. Even plant biologists interested to pursue their research on plant TLRs as Gram negative bacterial infection causes a great loss to agriculture, are facing the same problem. To pursue TLR4 research in all the organisms apart from fruit fly to humans, TLR4 antibodies can't be prepared by the conventional method because genome sequence for all the organisms is yet to be deciphered. As susceptibility to Gram negative bacterial infection and endotoxemia varies from species to species, study of species specific three dimensional crystallographic structure, sequence and protein folding of TLR4 is required to understand the mechanism for which functionally folded TLR4 from the organism need to be isolated. Species specific variation and expression can't be studied by isolating a fusion protein. By this simple method described here TLR4 from any species can be isolated easily and anti-TLR4 antibody can be prepared. Not only TLR4 but also other TLRs and scavenger receptors having a known directly binding ligand can also be isolated by this method.

Efficiency of anti-TLR4 antibodies is well documented in blocking Gram negative bacterial infection and sepsis. Anti-TLR4 antibodies (Roger et al, 2009, Spiller et al, 2008) and anti-TLR4/MD2 antibodies (Daubeuf et al, 2007) were demonstrated to block LPS induced signaling and protected from Gram negative bacterial infection and death. Another study reported anti-TLR4/MD2 antibodies offers protection against TNF-α induced hepatitis (Akashi et al, 2006). TLR2 driven septic shock by PAM₃CSK or B. subtilis infection could be blocked by anti-TLR2 antibodies (Meng et al, 2004). Furthermore it was demonstrated that administration of soluble TLR4 also blocks LPS signaling (Roger et al, 2009). In this study we developed polyclonal antibodies against the affinity purified receptor which protected mice from lethal endotoxic shock. Gram negative bacterial infection is a very common and an alarming problem in all the organisms from lower plants to human beings and TLR4 remain conserved in all the organisms, application of soluble TLR4 or anti-TLR4 antibodies may be a useful which can be prepared by this simple method. Considering all the above applications we concede the method of TLR4 isolation by affinity purification may be a useful tool in TLR research by which TLR from any species can be isolated in native form.

1. A method of isolating TLR4 from cell lysates of mononuclear cells comprising:
collecting bovine adult filarial parasites (Setaria digitata);
preparing aqueous extracts of Setaria digitata (Fag) to produce affinity purified filarial glycoprotein (AgW);
isolating human peripheral blood mononuclear cells (PBMCs) from heparinised venous blood samples;
subjecting the isolated human PBMCs to the step of incubation to produce PBMC lysates;
coupling the AgW with cyanogen bromide (CNBR) activated sepharose;
loading the AgW coupled with sepharose to an affinity purification column;
incubating PBMC lysates in the loaded affinity purification column to isolate an AgW recognizing receptor;
incubating the AgW recognizing receptor with rabbit anti-AgW recognizing receptor antibodies and rabbit antihuman TLR4 antibodies; and
detecting binding of the AgW recognizing receptors to the antibodies.

2. The method as claimed in claim 1, wherein Fag is prepared by homogenization followed by ultrasonication.

3. The method as claimed in claim 1, wherein AgW is biotinylated.

4. The method as claimed in claim 1, wherein the step of isolation of PBMC is done by a density gradient centrifugation method using Hisopaque.

5. The method as claimed in claim 1, wherein the PBMC are incubated with 2 ml of cell lysis solution and a cocktail of protease inhibitors for 1 hour at 4° C. and then ultrasonicated.

6. The method as claimed in claim 1, wherein human PBMC lysates are incubated in the column at 37° C. for 30 minutes and then the column is washed by PBS to remove the unbound proteins and the proteins bound to AgW in the column are eluted using glycine HCl buffer.

7. The method as claimed in claim 1, wherein the anti-AgW recognizing receptor antibodies in rabbits are prepared by immunizing a rabbit with 0.3 mg of AgW recognizing receptor with complete Freunds Adjuvant subcutaneously.

8. The method as claimed in claim 3, wherein the AgW is biotinylated with an N-hydroxysuccinamide derivative.
